(19) United States
(12) Patent Application Publication
(10) Pub. No.: US 2015/0380655 A1
(43) Pub. Date: Dec. 31, 2015

(54) ANTHRACENE DERIVATIVE, METHOD FOR PREPARING THE SAME, USE THEREOF AND ORGANIC LIGHT EMITTING DEVICE

(71) Applicants: BOE TECHNOLOGY GROUP CO., LTD., Beijing (CN); JILIN OLED MATERIAL TECH CO., LTD., Changchun City, Jilin Province (CN)

(72) Inventors: Peng Zhang, Beijing (CN); Xiaoyu Ma, Beijing (CN); Hui Wang, Beijing (CN); Lujiang Huangfu, Beijing (CN)

(73) Assignees: BOE TECHNOLOGY GROUP CO., LTD., Beijing (CN); JILIN OLED MATERIAL TECH CO., LTD., Changchun City, Jilin Province (CN)

(21) Appl. No.: 14/416,957

(22) PCT Filed: May 29, 2014
(86) PCT No.: PCT/CN2014/078772

§ 371 Date: Jan. 23, 2015

(30) Foreign Application Priority Data
Dec. 10, 2013 (CN) 201310670362.7

(51) Int. Cl. H01L 51/00; C07C 17/02; C09K 11/06; C07C 15/28; C07C 1/30

(52) U.S. Cl. CPC H01L 51/0058; C07C 15/28; C07C 1/30; C09K 11/06; C07C 17/02; C09K 2211/1011; H01L 51/5012

(57) ABSTRACT

Provided are anthracene derivative, method for preparing the same, use thereof, and an organic light emitting device. The anthracene derivative represented by a formula:

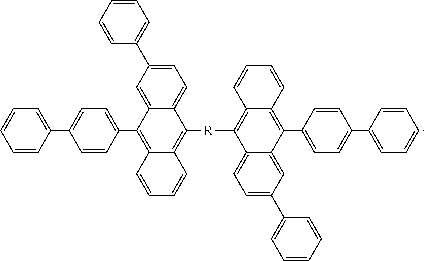

### CROSS-REFERENCE TO RELATED APPLICATION

This application is the U.S. national phase of PCT Application No. PCT/CN2014/078772 filed on May 29, 2014, which claims priority to Chinese Patent Application No. 201310670362.7 filed on Dec. 10, 2013, the disclosures of which are incorporated in their entirety by reference herein.

### TECHNICAL FIELD

The present disclosure relates to an anthracene derivative, a method for preparing the same, a use thereof, and an organic light emitting device.

### BACKGROUND

An organic electroluminescent device generally consists of a pair of opposing electrodes and at least one layer of an organic light emitting compound inserted therebetween. Charges are injected into the layer of the organic light emitting compound formed between the anode and the cathode, so as to form pairs of electrons and holes, which allow an organic compound having fluorescent or phosphorescent property to emit lights.

A Study on an organic electroluminescent material has been started from 1950, when Bernose observed a light emitting phenomenon by applying a high voltage to a high molecule thin film containing an organic pigment. In 1965, Pope and et al. firstly discovered electroluminescent property of an anthracene compound.

In order to manufacture an organic light emitting device with a high efficiency, researchers have gradually changed a structure of an organic layer in the device from a single layer to plural layers. The organic electroluminescent device being designed with the plural layers is because a transport efficiency between the electrons and the holes may be improved by appropriately designing a hole injection layer, a hole transport layer an electron transport layer and an electron injection layer based on different moving speed of the holes and the electrons, so that the holes and the electrons may achieve a balance, to improve a light emitting efficiency.

In 1987, Tang and et al. from Kodak Company found that by using ITO as an anode, a Mg—Ag alloy as a cathode, tri-(8-hydroxyquinolinato)aluminium (Alq3) as an electron transport material and a light emitting material, a triphenylamine derivative as a hole transport material, a separation functional organic light emitting device (OLED) having a double-layer structure emits lights with an intensity of about 1000 cd/m² when being applied with about 10V of a voltage. The OLED has a stack structure of the electron transport material and the hole transport material, and has an improved light emitting property comparing with a traditional light emitting device having a single-layer structure. Such study indicates that a display with a high brightness and a high efficiency may be researched and developed using an organic thin film light emitting diode, which attracts a worldwide attention and plays a major role in future research of OLED.

From 1980, a general structure of the OLED is a simple structure including an anode (ITO), a hole transport layer (HTL), an emitting layer (EML), a cathode (Mg:Ag). After then, a study on a hole injection layer (such as phthalocyanine copper (CuPc)), an electron injection layer (LiF), and a cathode (Al:Li) has started. As a large number of the organic layers are inserted, the structure of the OLED device became complex, by which difficulty is increased technically. However, in order to reduce the number of manufacturing steps and improve the power efficiency in production, it is desirable to reduce the number of layers.

### SUMMARY

In view of the above-described problems, the present disclosure provides a novel asymmetric anthracene derivative, which can be used as a host material, a hole injection material or a hole transport material. The anthracene derivative according to the present disclosure may improve a light emitting efficiency and a light emitting brightness, which may greatly improve properties of an organic electroluminescent device in various aspects.

Technical solutions of the present disclosure are shown as below:

An anthracene derivative represented by a formula:

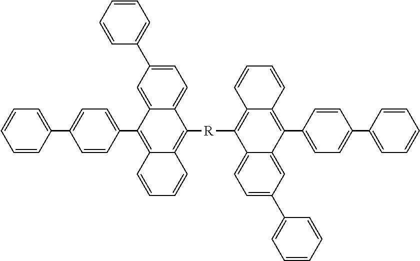

wherein R is selected from substituted or unsubstituted C6-C32 aryl.

Specifically, R may be any one selected from a, b, c, d, e and f,

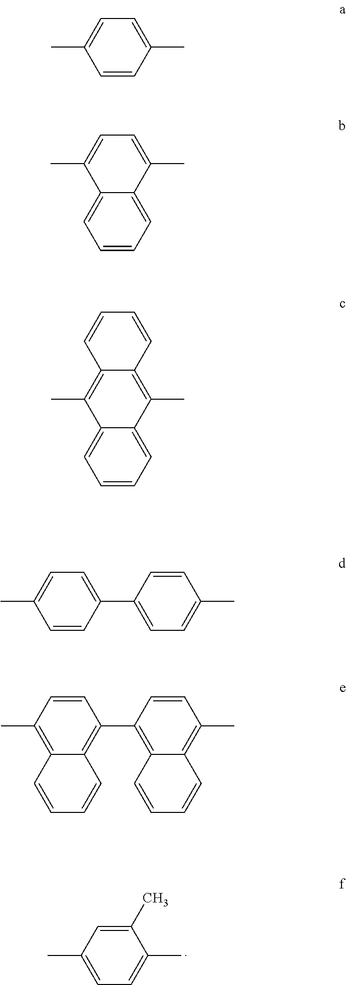

Specifically, the anthracene derivative may be any one selected from compounds represented by formulas 001 to 006:

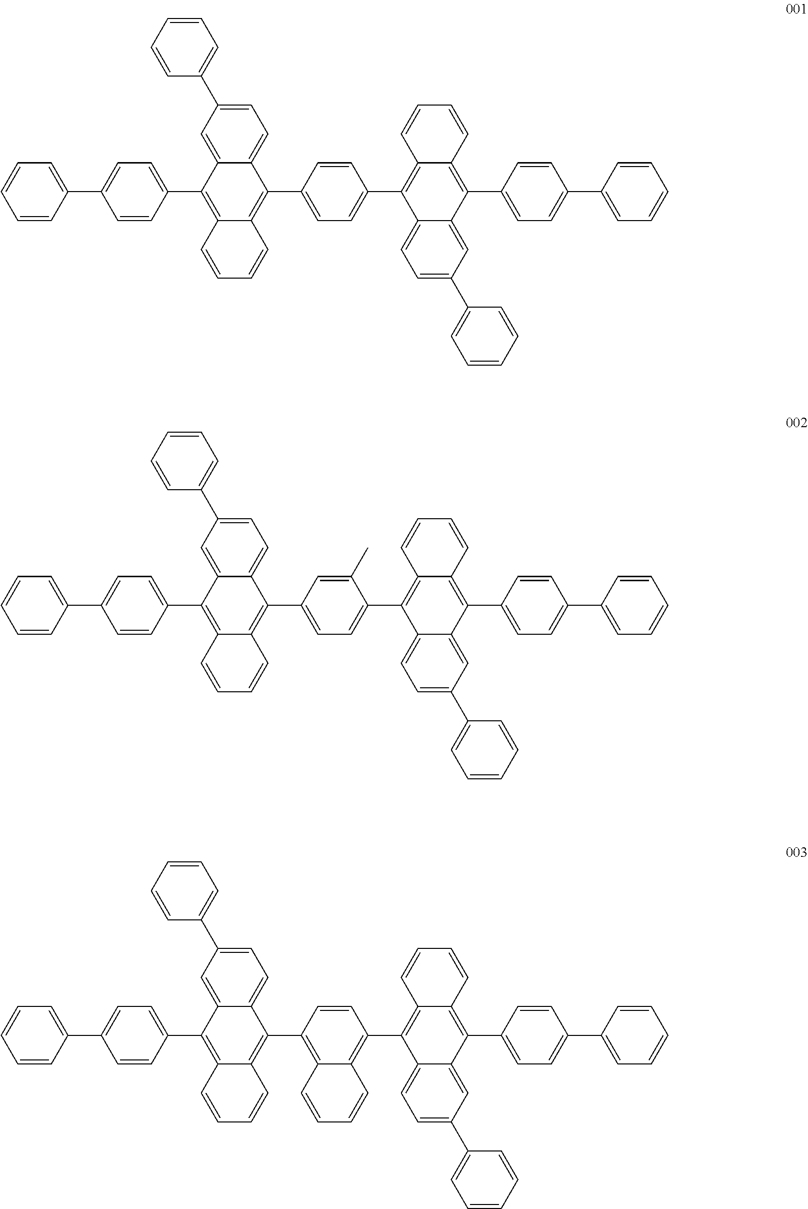

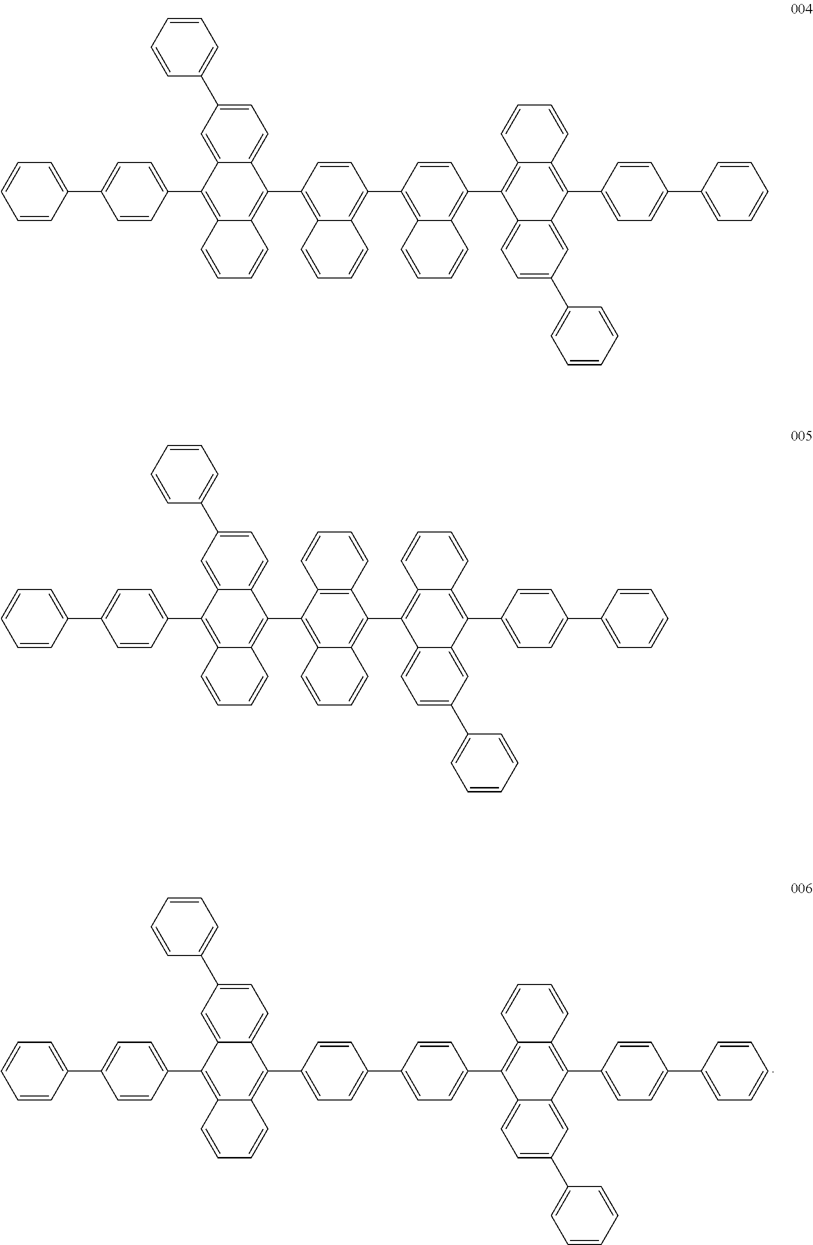

A method for preparing the anthracene derivative according to the present disclosure, includes the following steps:

step S1: adding

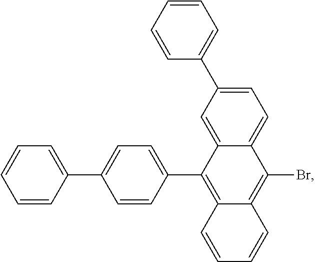 [SMILES: Brc1c2ccccc2c(-c2ccc(-c3ccccc3)cc2)c2cc(-c3ccccc3)ccc12]

a substituted or unsubstituted aryl diboric acid compound, potassium carbonate and methylbenzene into a degassed reaction vessel;

step S2: increasing a temperature of the reaction vessel to 70° C., adding a catalyst and refluxing for reacting sufficiently; and

step S3: adding a terminator, filtering, washing a recrystal product, to obtain the anthracene derivative.

Alternatively, in the step S1,

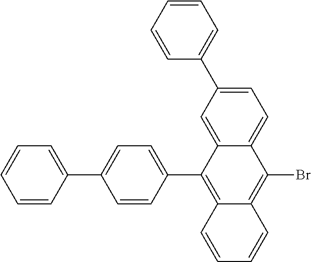 [SMILES: Brc1c2ccccc2c(-c2ccc(-c3ccccc3)cc2)c2cc(-c3ccccc3)ccc12]

is obtained by the following steps:

step N1: adding

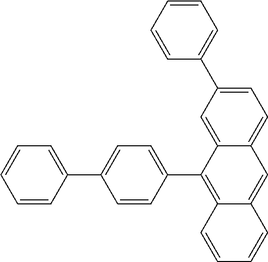 [SMILES: c1ccc(-c2ccc(-c3c4ccccc4cc4ccc(-c5ccccc5)cc34)cc2)cc1]

and a solvent into the degassed reaction vessel;

step N2: increasing a temperature of reaction system to 60° C. for reacting sufficiently; and

N3: filtering and washing to obtain

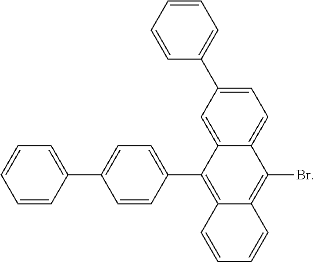 [SMILES: Brc1c2ccccc2c(-c2ccc(-c3ccccc3)cc2)c2cc(-c3ccccc3)ccc12]

Alternatively, the substituted or unsubstituted aryl diboric acid compound may be at least one selected from followings:

terephthalic acid,

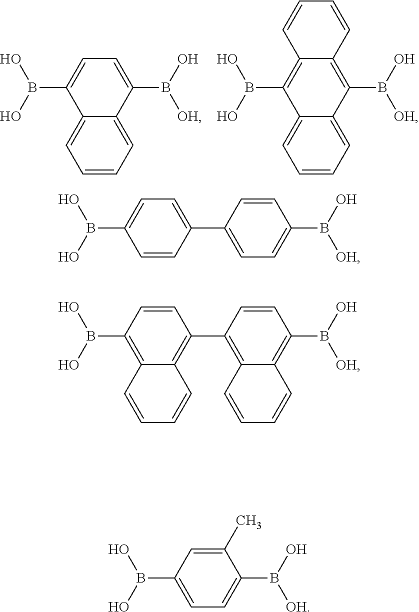

The anthracene derivative according to the present disclosure can be used as a fluorescence host material, a hole injection material or a hole transport material in an organic electroluminescent device.

Specifically, the anhracene derivative is used as a fluorescent green host material in the organic electroluminescent device.

The anthracene derivative according to the present disclosure may be used in manufacturing an organic light emitting device. The organic light emitting device includes a first electrode, a second electrode, and one or more organic material layers between the first electrode and the second electrode, wherein at least one of organic compound layer includes the anthracene derivative.

The anthracene derivative according to the present disclosure has a high light emitting efficiency, indicating that such compound may be used as a light emitting material or a light emitting host material; particularly the compound may be used as a fluorescence host material. The anthracene derivative also has a high glass transition temperature being difficult to be crystallized, and may be used in the organic electroluminescent device, exhibiting a higher efficiency, a higher brightness, a longer product life and a better charge transportability, so that the organic electroluminescent device may have a prolonged product life and a decreased production cost.

### DETAILED DESCRIPTION

The present disclosure provides an anthracene compound, a method for preparing the same and a use thereof To make the objects, the technical solutions and the advantages of the present disclosure more clearly and apparent, the technical solutions of the present disclosure are further described specifically. It should be understood that specific examples described herein are only used to explain the present disclosure, but not intended to limit the present disclosure.

The present disclosure provides an anthracene compound represented by a formula:

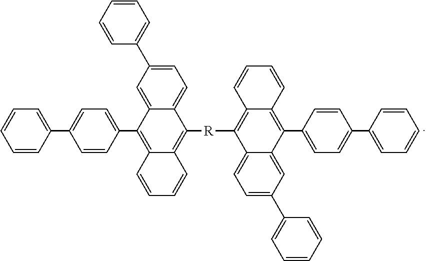

in which, R is selected from substituted or unsubstituted C6-C32 aryl.

Specifically, R may be any one selected from a, b, c, d, e and f,

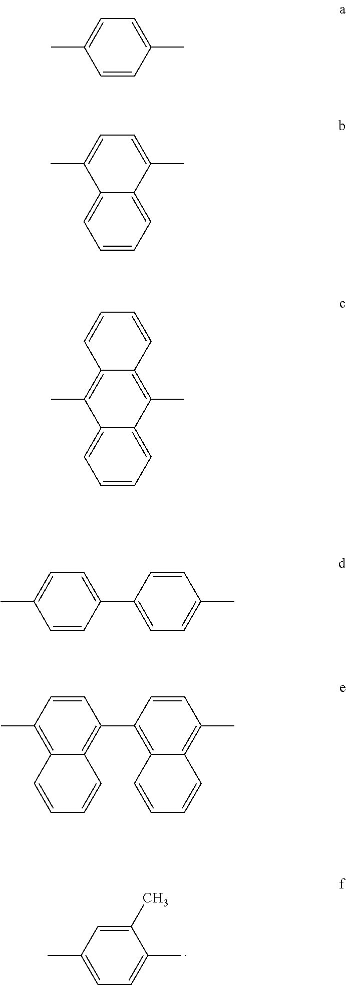

Specifically, the anthracene derivative may be any one selected from compounds represented by formulas 001 to 006:

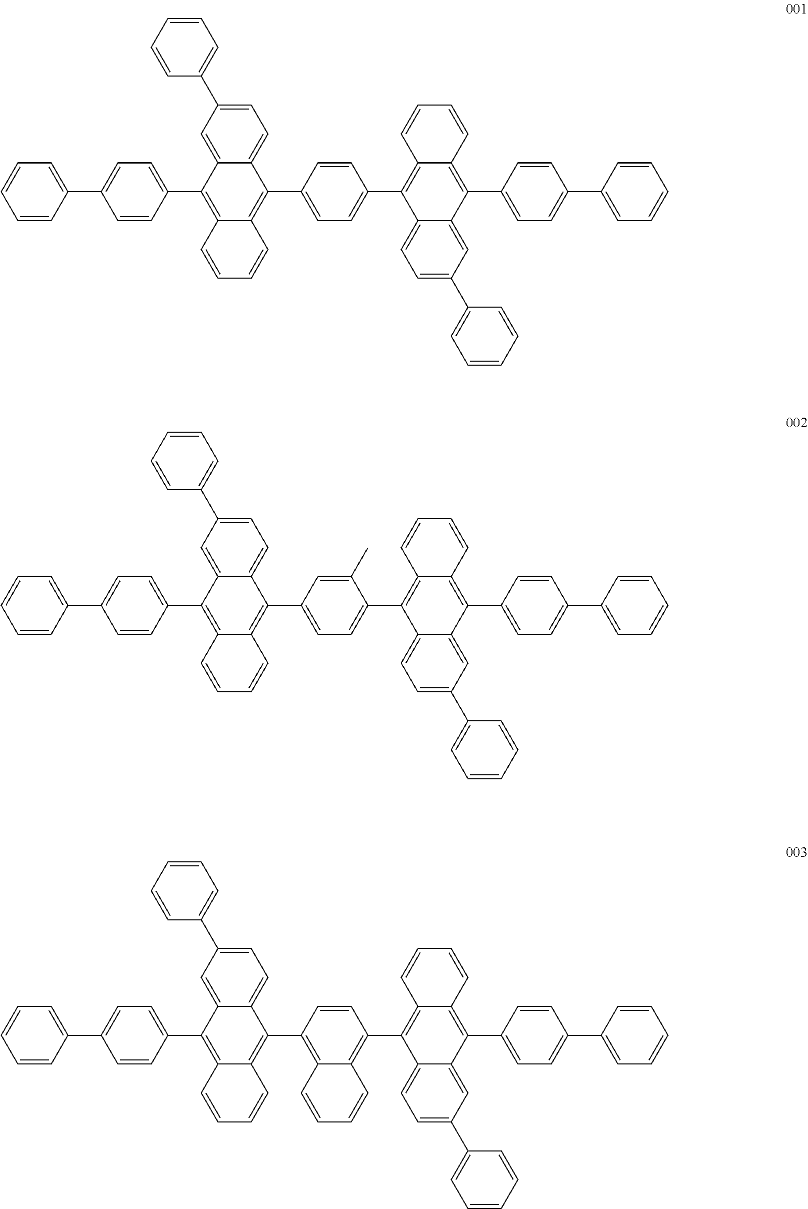

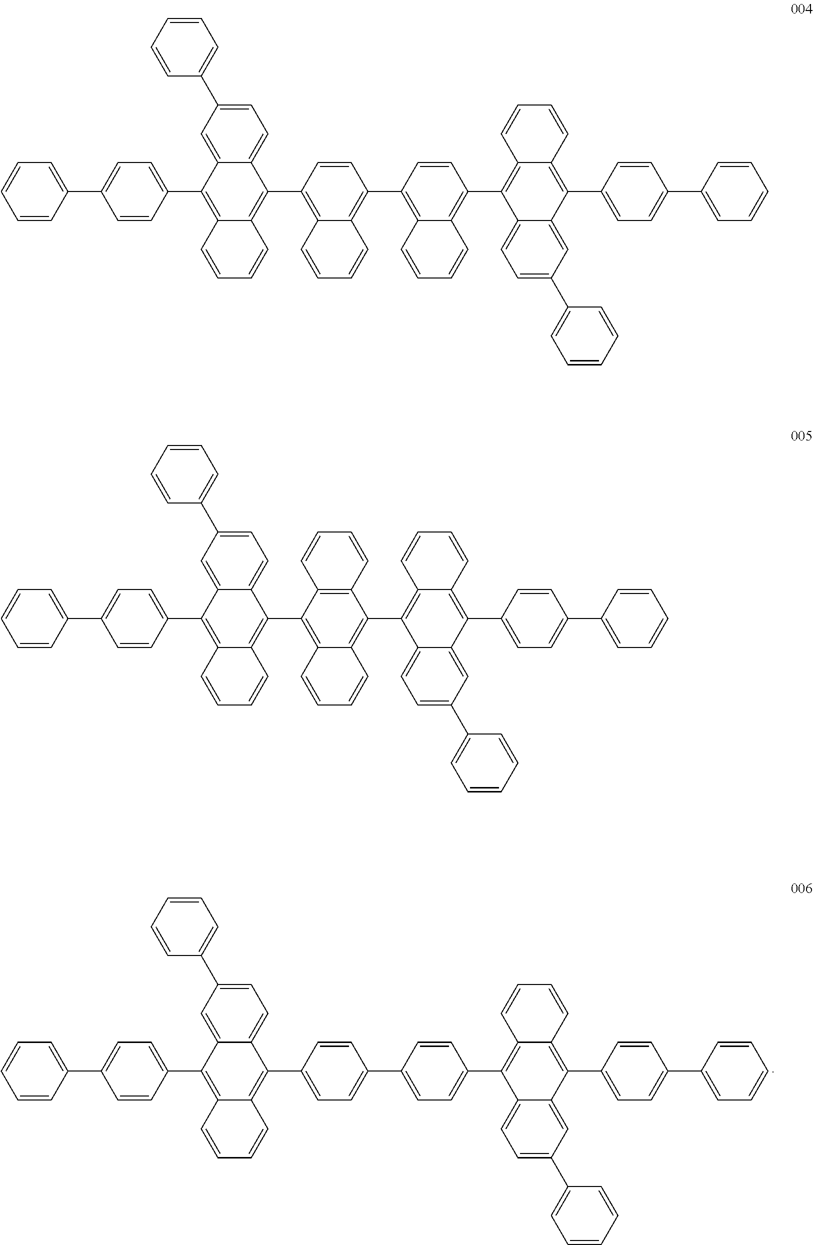

A method for preparing the anthracene derivative according the present disclosure, includes the following steps:

step S1: adding

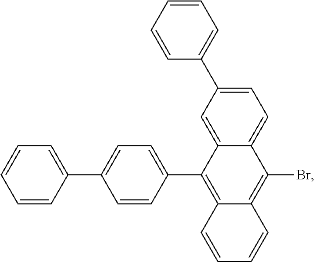 [SMILES: Brc1c2ccccc2c(-c2ccc(-c3ccccc3)cc2)c2cc(-c3ccccc3)ccc12]

a substituted or unsubstituted aryl diboric acid compound, potassium carbonate and methylbenzene into a degassed reaction vessel;

step S2: increasing a temperature of the reaction vessel to 70° C., adding a catalyst and refluxing for reacting sufficiently; and

step S3: adding a terminator, filtering, washing a recrystal product, to obtain the anthracene derivative.

In which, in the step S1,

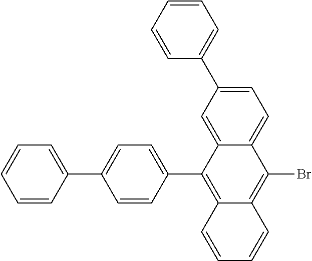 [SMILES: Brc1c2ccccc2c(-c2ccc(-c3ccccc3)cc2)c2cc(-c3ccccc3)ccc12]

is obtained by the following steps:

step N1: adding

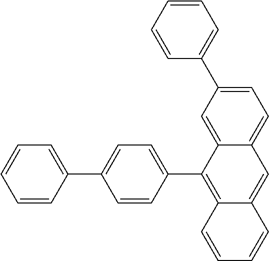 [SMILES: c1ccc(-c2ccc(-c3c4ccccc4cc4ccc(-c5ccccc5)cc34)cc2)cc1]

and a solvent into the degassed reaction vessel;

step N2: increasing a temperature of reaction system to 60° C. for reacting sufficiently; and

step N3: filtering and washing to obtain

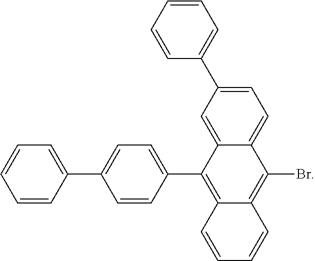 [SMILES: Brc1c2ccccc2c(-c2ccc(-c3ccccc3)cc2)c2cc(-c3ccccc3)ccc12]

Alternatively, the substituted or unsubstituted aryl diboric acid compound may be at least one selected from followings:

terephthalic acid,

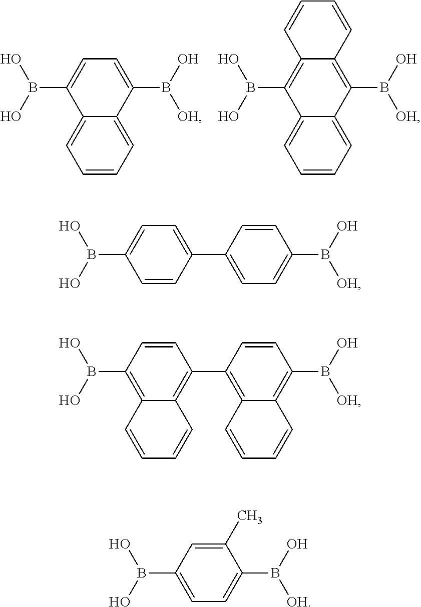 [SMILES: OB(O)c1ccc(B(O)O)c2ccccc12 ; OB(O)c1c2ccccc2c(B(O)O)c2ccccc12 ; OB(O)c1ccc(-c2ccc(B(O)O)cc2)cc1 ; OB(O)c1ccc(-c2ccc(B(O)O)c3ccccc23)c2ccccc12 ; Cc1cc(B(O)O)ccc1B(O)O]

Specifically, in order to describe the method for preparing the anthracene derivative of the present disclosure in detail, an anthracene compound

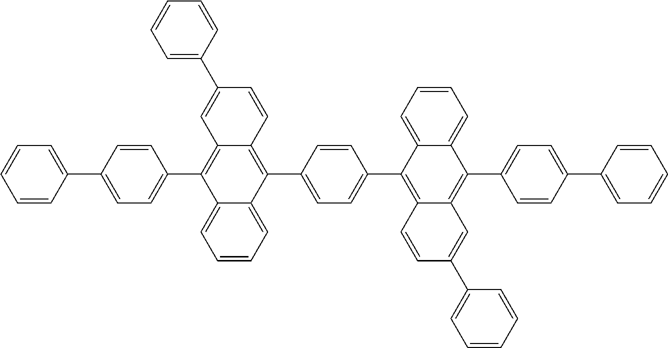 [SMILES: c1ccc(-c2ccc(-c3c4ccccc4c(-c4ccc(-c5c6ccccc6c(-c6ccc(-c7ccccc7)cc6)c6cc(-c7ccccc7)ccc56)cc4)c4ccc(-c5ccccc5)cc34)cc2)cc1]

having a formula 001 is taken as an example for description. A detailed reaction equation is shown as below

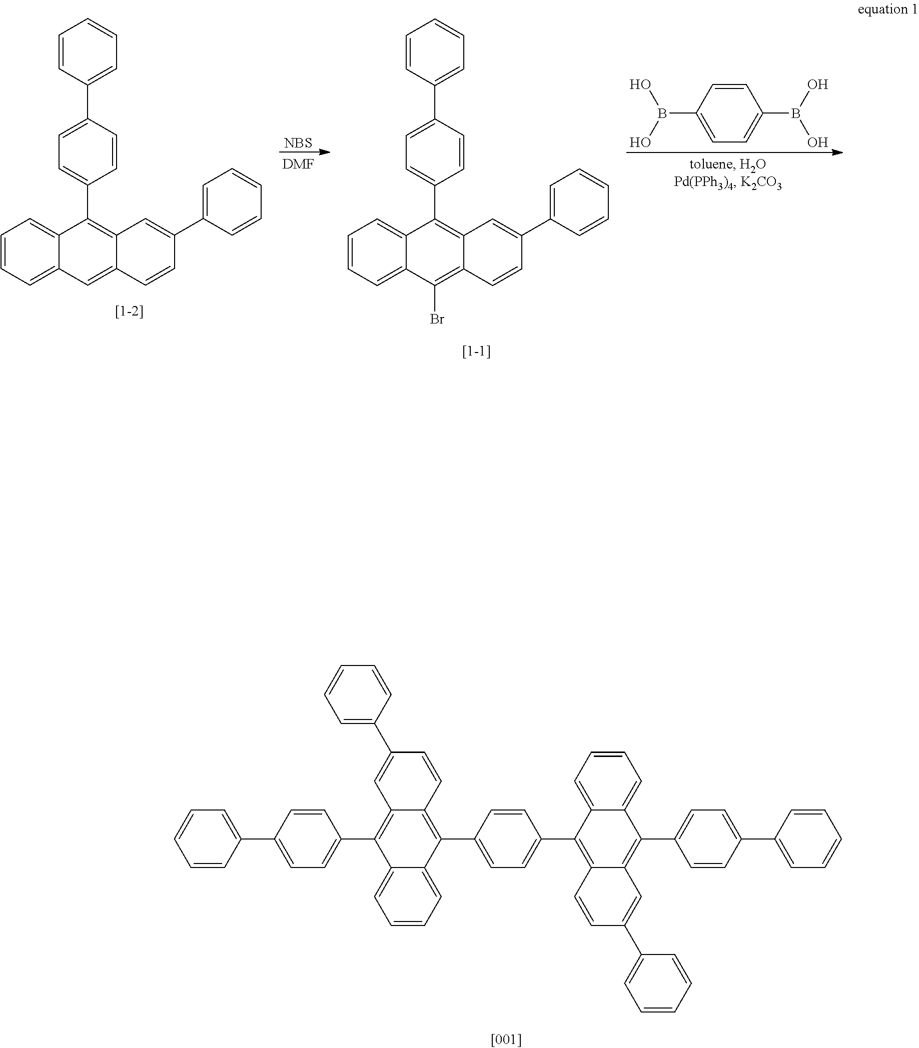

Compound [1-2] (86 g, 0.21 mol), NBS (N-bromobutanimide) (49.83 g, 0.28 mol)and 1.2 L of DMF (N,N-dimethylformamide) were added into a first reaction kettle having a volume of 2 L under a protection of nitrogen atmosphere.

After a temperature of the first reaction kettle was increased to 60° C., a reaction therein was allowed for 16 hours under a stirring condition. A method of thin layer chromatography (TLC) was used to confirm whether the reaction was completed.

After a filtration under a vacuum atmosphere, 500 mL of acetone was used to obtain a suspension, which was stirred under commutation. After being filtered under a vacuum atmosphere, 85.7 g of target compound [1-1] was obtained, being as a solid having a light green color, with a yield of 83%.

Compound [1-1] (29.7 g, 0.066 mol), p-benzenediboronic acid (4.97 g, 0.03 mol), K₂CO₃ (9.12 g, 0.066 mol) and 200 mL of methylbenzene were added into a second reaction kettle having a volume of 2 L under a protection of nitrogen atmosphere, and mixed by being stirred.

After being heated to 70° C., the second reaction kettle was added with Pd(PPh₃)₄ (0.35 g, 0.0003 mol) and 100 mL of distilled water, which were then stirred and refluxed for 11 hours for a sufficient reaction.

After the reaction was stopped by adding 70 mL of distilled water, a filtration under a vacuum atmosphere was performed to obtain a solid, which was washed using distilled water and then recrystallized using actone, methylbenzene and THF, and recrystallized solid was subsequently sublimated and recrystallized again, to obtain 18.36 g of target compound 001, being as a milky solid with a yield of 69%.

Compounds 001 to 006 were synthesized according to the above exemplary method (in which, p-benzenediboronic acid was replaced by

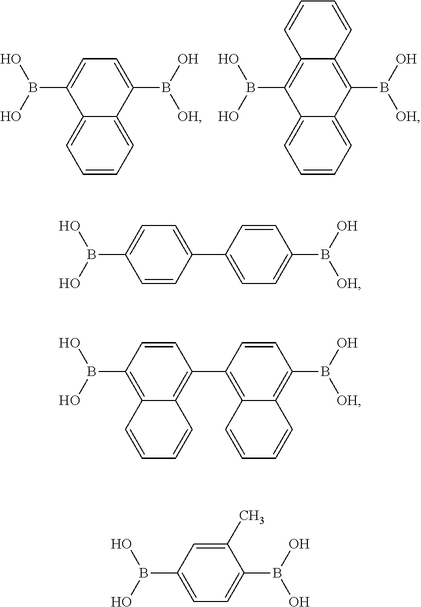 [SMILES: OB(O)c1ccc(B(O)O)c2ccccc12 ; OB(O)c1c2ccccc2c(B(O)O)c2ccccc12 ; OB(O)c1ccc(-c2ccc(B(O)O)cc2)cc1 ; OB(O)c1ccc(-c2ccc(B(O)O)c3ccccc23)c2ccccc12 ; Cc1cc(B(O)O)ccc1B(O)O]

respectively for preparing compounds 001 to 006). The results were shown in Table 1:

TABLE 1

| Compound | | |
|---|---|---|
| No. | Elements analysis | MS/FAB(M⁺) |
| 001 | calculating value--C: 94.77%; H: 5.23%; | 887.11 |
| | testing value --C: 94.76%; H: 5.24%; | |
| 002 | calculating value--C: 94.63%; H: 5.37%; | 901.14 |
| | testing value--C: 94.62%; H: 5.38%; | |
| 003 | calculating value--C: 94.84%; H: 5.16%; | 937.17 |
| | testing value--C: 94.85%; H: 5.15%; | |
| 004 | calculating value--C: 94.88%; H: 5.12%; | 1063.33 |
| | testing value-C: 94.86%; H: 5.14%; | |
| 005 | calculating value--C: 94.90%; H: 5.10%; | 987.23 |
| | testing value--C: 94.92%; H: 5.08%; | |
| 006 | calculating value--C: 94.77%; H: 5.23%; | 963.21 |
| | testing value--C: 92.47%; H: 4.91%; | |

The present disclosure is described in further detail with reference to following examples. However, it should be understood that examples described hereinafter are only used to explain the present disclosure, but not intended to limit the scope of the present disclosure. Within the scope of the present disclosure, the examples could be revised or changed according to requirements of implementers.

Specifically, the present disclosure is described below by taking a fluorescent green host material as an example.

### COMPARATIVE EXAMPLE 1

Hereinafter, an organic light emitting device with following structure was manufactured as the comparative sample 1 by using compound a as a fluorescent green host material, using compound b as a fluorescent green dopped material, using 2-TNATA as a hole injection layer material, using α-NPD (N,N′-dinaphthyl-N,N′-diphenybenzidine) as a hole transport layer material. The organic light emitting device had a structure of ITO/2-TNATA (80 nm)/α-NPD (30 nm)/compound a+compound b (30 nm)/Alq₃ (30 nm)/LiF (0.5 nm)/Al (60 nm).

After being cut into a size of 50 mm* 50 mm* 0.7 mm, 15 Ω/cm² (1000 Å) of an ITO glass substrate purchased from Corning Company, was washed under microwave using acetone, isopropanol, purified water for 15 min respectively, and then washed in UV for another 30 min. A layer of 2-TNATA having a thickness of 80 nm was deposited on obtained substrate by a vacuum evaporation to form a hole injection layer. And a layer of α-NPD having a thickness of 30 nm was deposited on the hole injection layer by a vacuum evaporation to form a hole transport layer. Then a layer of compound a and compound b (doping ratio: 3%) having a thickness of 30 nm was deposited on the hole transport layer by a vacuum evaporation to form a light emitting layer having a thickness of 30 nm. And then the organic light emitting device was manufactured by depositing 0.5 nm of LiF (electron injection layer) and 60 nm of Al on the electron transport layer by a one-time vacuum evaporation.

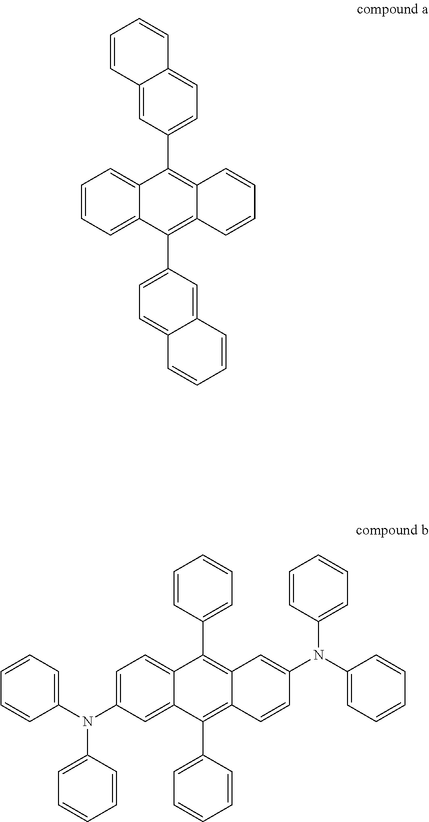

### Examples 1 to 6

Organic light emitting devices with following structures: ITO/2-TNATA (80 nm)/α-NPD (30 nm)/[one of the fluorescent green host compound 001 to 006]/b(3%)/(30 nm)/Alq3 (30 nm)/LiF (0.5 nm)/Al (60 nm), were manufactured using a method same as that in the comparative example 1, except the compound a being as the fluorescent host material for the light emitting layer was replaced by compound 001 to 006 respectively, by which samples 1 to 6 were obtained accordingly.

The light emitting properties of the comparative sample 1 and the samples 1 to 6 were measured.

A driving voltage, a brightness, a light emitting efficiency, a light color were evaluated using Keithley SMU235, PR650. The same tests were carried out with the comparative sample 1 and the samples 1 to 6, and obtained results were shown in Table2.

TABLE 2

| | Host | Dopped | Bright- | Light emitting | Wave- |
|---|---|---|---|---|---|
| | com- | com- | ness | efficiency | length |
| No. | pounds | pounds | [cd/m²] | [cd/A] | [nm] |
| Comparative | a | b | 2032 | 20.3 | 516 |
| Sample 1 | | | | | |
| Sample 1 | 001 | b | 2369 | 23.7 | 517 |
| Sample 2 | 002 | b | 2326 | 23.3 | 518 |
| Sample 3 | 003 | b | 2348 | 23.5 | 522 |
| Sample 4 | 004 | b | 2437 | 24.4 | 524 |
| Sample 5 | 005 | b | 2431 | 24.1 | 519 |
| Sample 6 | 006 | b | 2219 | 22.2 | 520 |

As shown in Table 2, comparing with the comparative sample 1, the samples 001 to 006 shows a color of emitting lights being as green within a wavelength ranging from 516 nm to 524 nm. The light emitting efficiency and the brightness of the samples 1 to 6 are significantly improved.

It should be noted that, a person skilled in the art may further make improvements and modifications without departing from the principle of the present disclosure, and these improvements and modifications shall also be considered as the scope of the present disclosure.

What is claimed is:

1. An anthracene derivative represented by a formula: 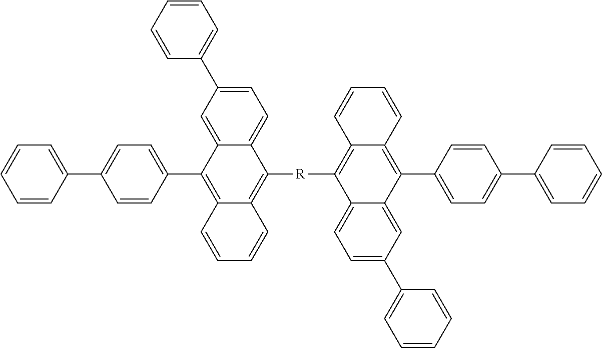
wherein R is selected from substituted or unsubstituted C6-C32 aryl.

2. The anthracene derivative according to claim 1, wherein R is any one selected from a, b, c, d, e and f,

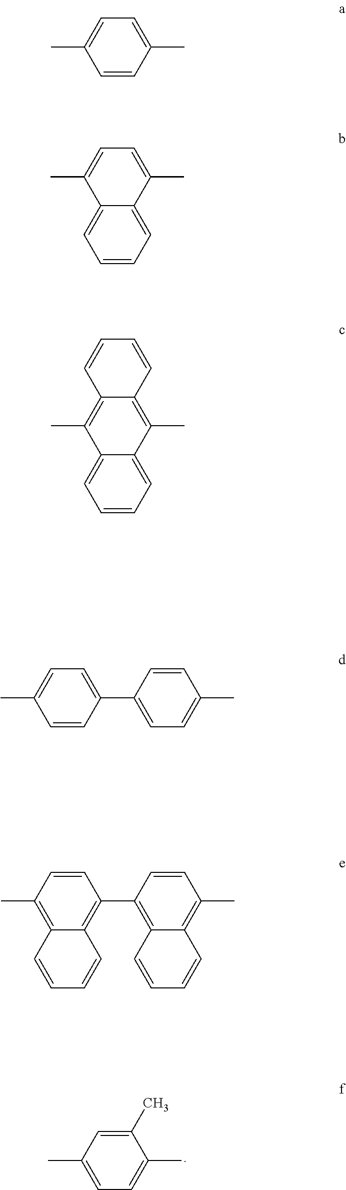

3. The anthracene derivative according to claim 1, wherein the anthracene derivative is any one selected from compounds represented by formulas 001 to 006:

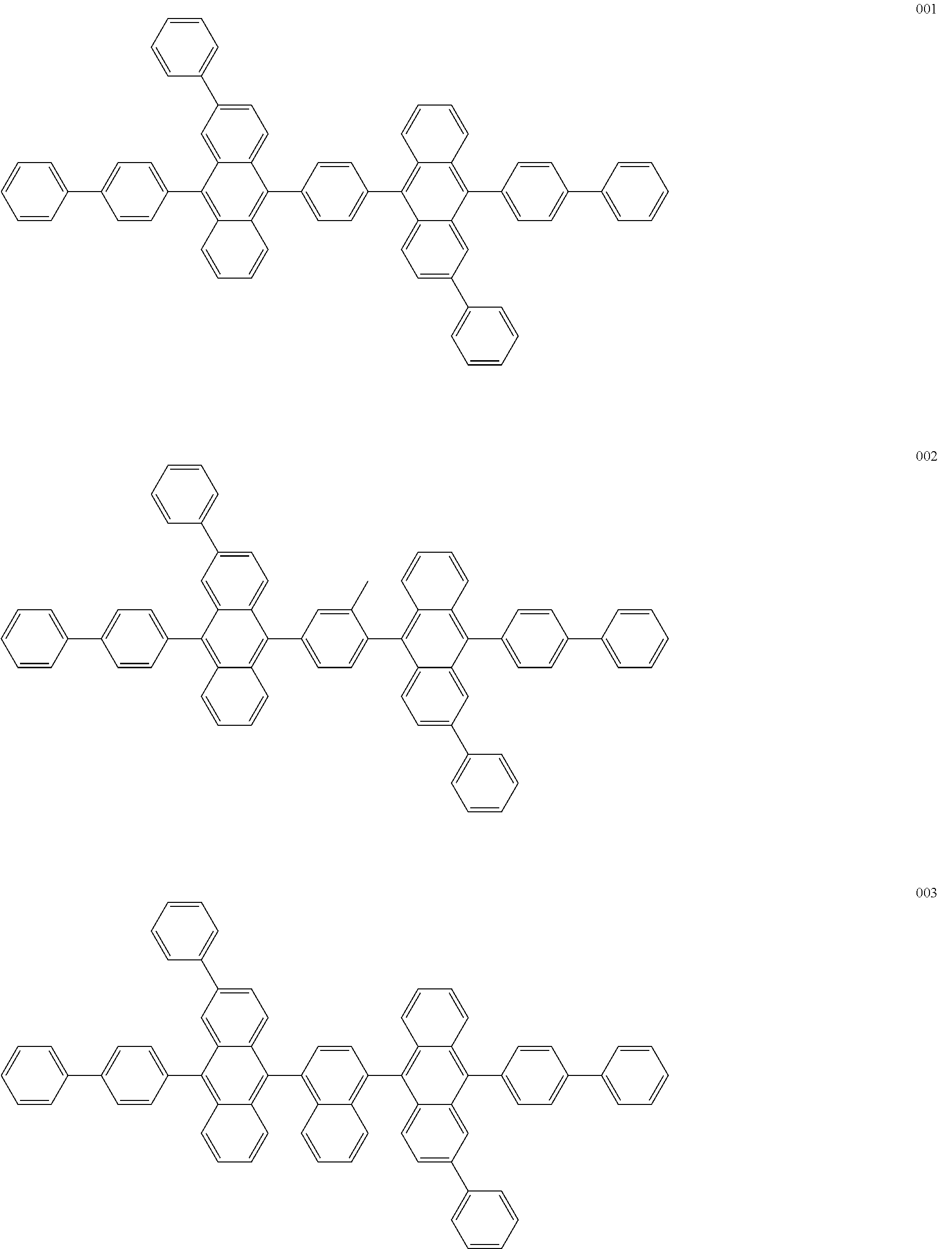

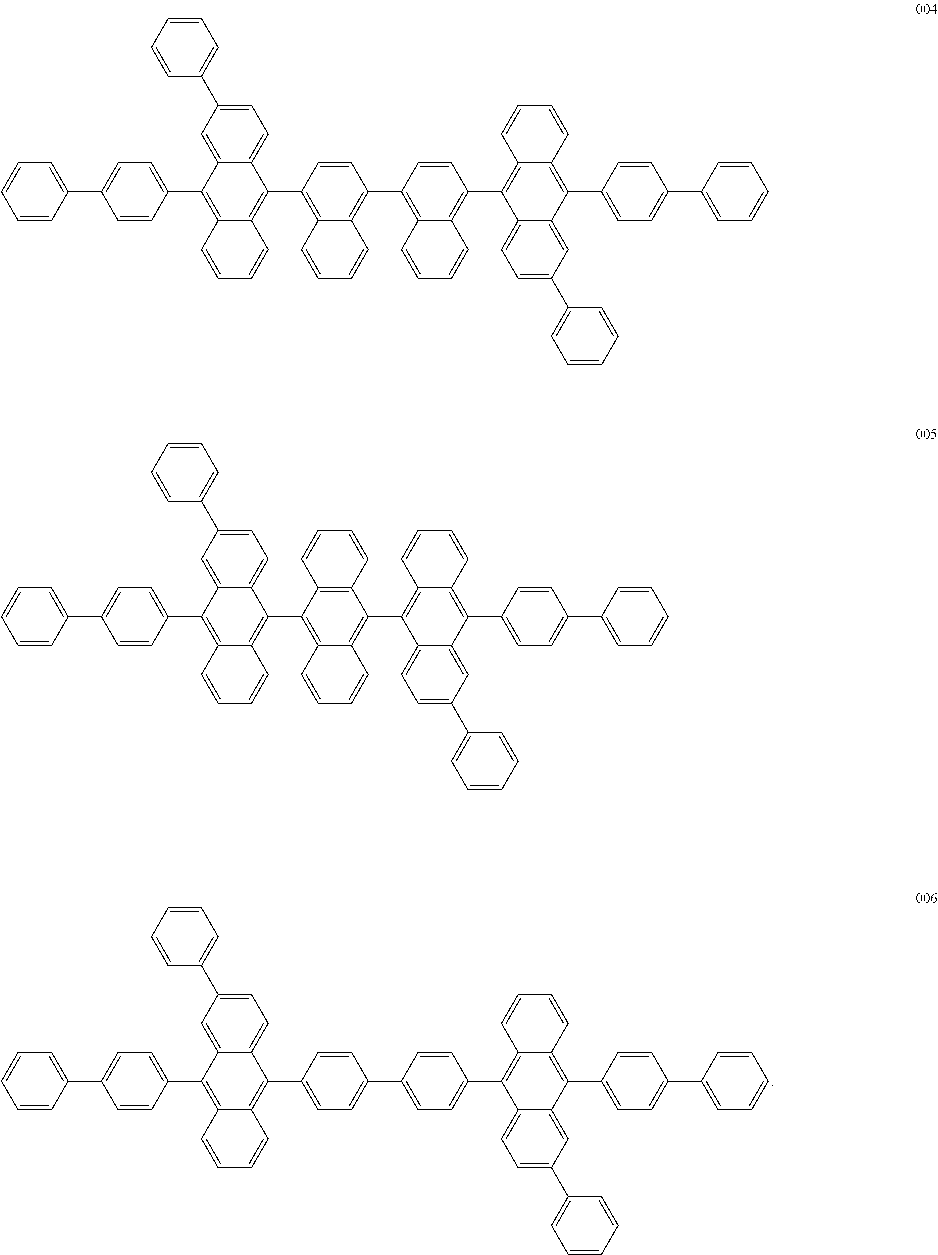

4. A method for utilizing the anthracene derivative according to claim 1 in an organic electroluminescent device, comprising: using the anthracene derivative as a fluorescence host material, a hole injection material or a hole transport material in the organic electroluminescent device.

5. The method according to claim 4, wherein the anthracene derivative is used as a fluorescent green host material in the organic electroluminescent device.

6. An organic light emitting device, comprising:
a first electrode;
a second electrode; and
one or more organic compound layers between the first electrode and the second electrode,
wherein at least one organic compound layer comprises the anthracene derivative according to claim 1.

7. A method for preparing the anthracene derivative according to claim 1, comprising the following steps:
step S1: adding 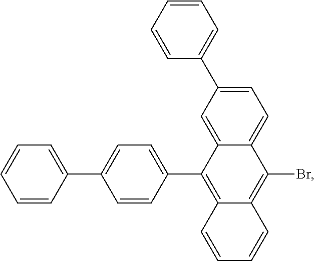 [SMILES: Brc1c2ccccc2c(-c2ccc(-c3ccccc3)cc2)c2cc(-c3ccccc3)ccc12]
a substituted or unsubstituted aryl diboric acid compound, potassium carbonate and methylbenzene into a degassed reaction vessel;
step S2: increasing a temperature of the reaction vessel to 70° C., adding a catalyst and refluxing for reacting sufficiently; and
step S3: adding a terminator, filtering, washing a recrystal product, to obtain the anthracene derivative.

8. The method according to claim 7, wherein in the step S1, 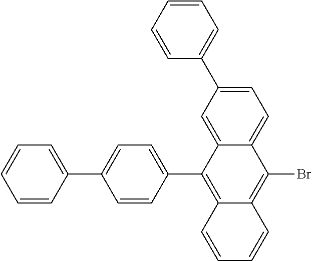 [SMILES: Brc1c2ccccc2c(-c2ccc(-c3ccccc3)cc2)c2cc(-c3ccccc3)ccc12]
is obtained by the following steps:
step N1: adding 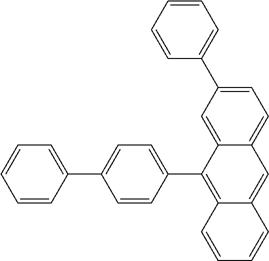 [SMILES: c1ccc(-c2ccc(-c3c4ccccc4cc4ccc(-c5ccccc5)cc34)cc2)cc1]
and a solvent into the degassed reaction vessel;
step N2: increasing a temperature of a reaction system to 60° C. for reacting sufficiently; and
step N3: filtering and washing to obtain

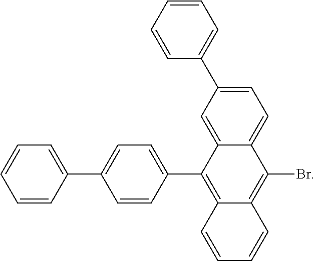 [SMILES: Brc1c2ccccc2c(-c2ccc(-c3ccccc3)cc2)c2cc(-c3ccccc3)ccc12]

9. The method according to claim 7, wherein the substituted or unsubstituted aryl diboric acid compound is at least one selected from followings:
terephthalic acid,

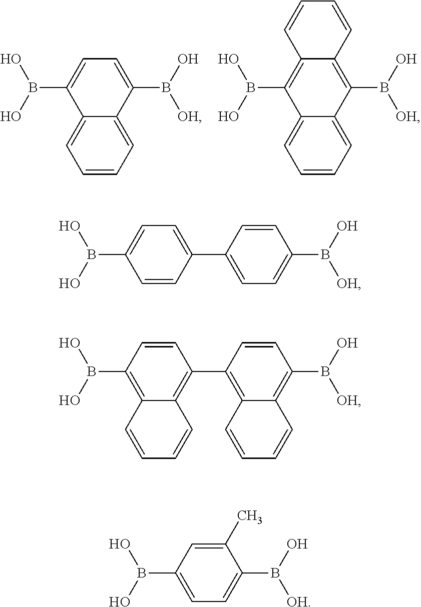 [SMILES: OB(O)c1ccc(B(O)O)c2ccccc12 ; OB(O)c1c2ccccc2c(B(O)O)c2ccccc12 ; OB(O)c1ccc(-c2ccc(B(O)O)cc2)cc1 ; OB(O)c1ccc(-c2ccc(B(O)O)c3ccccc23)c2ccccc12 ; Cc1cc(B(O)O)ccc1B(O)O]

10. An organic light emitting device, comprising:
a first electrode;
a second electrode; and
one or more organic compound layers between the first electrode and the second electrode,
wherein at least one organic compound layer comprises the anthracene derivative according to claim 2.

11. An organic light emitting device, comprising:
a first electrode;
a second electrode; and
one or more organic compound layers between the first electrode and the second electrode,
wherein at least one organic compound layer comprises the anthracene derivative according to claim 3.
